(19) United States
(12) Patent Application Publication
(10) Pub. No.: US 2002/0102258 A1
(43) Pub. Date: Aug. 1, 2002

(54) Human tumor necrosis factor receptor-like 2 (TR2) antibodies

(75) Inventors: Jeremy A. Harrop, Malvern, PA (US); Stephen D. Holmes, Epsom (GB); Manjula P. Reddy, Phoenixville, PA (US); Alemseged Truneh, West Chester, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/020,787

(22) Filed: Dec. 14, 2001

Related U.S. Application Data
(63) Continuation of application No. 09/403,815, filed on Oct. 26, 1999, now abandoned.
Application No. 09/403,815 is a 371 of international application No. PCT/US98/09744, filed on May 12, 1998.
(60) Provisional application No. 60/046,249, filed on May 12, 1997.

(51) Int. Cl. A61K 39/395

(52) U.S. Cl. 424/143.1

(57) ABSTRACT

The present invention relates to antibodies to novel members of the Tumor Necrosis Factor (TNF) receptor family called TR2 receptor and their uses in pathological conditions. Hybridoma cell lines producing such mAbs, methods of in vivo imaging of pathological conditions, and methods of treating and diagnosing pathological conditions, caused by abnormal functioning, production or metabolism of TR2 receptors are also provided. In vitro assays for detecting the presence of TR2 and for evaluating the binding affinity of a test compound are also described.

### BACKGROUND OF THE INVENTION

1. Field of the Invention

The present invention relates to antibodies to novel members of the Tumor Necrosis Factor (TNF) receptor family and their uses in pathological conditions.

2. Related Art

Human tumor necrosis factors alpha (TNF-alpha) and (TNF-beta or lymphotoxin) are related members of a broad class of polypeptide mediators, which includes the interferons, interleukins and growth factors, collectively called cytokines (Beutler, B. and Cerami, A., Annu. Rev. Immunol., 7:625-655 (1989)).

Tumor necrosis factor (TNF-alpha and TNF-beta) was originally discovered as a result of its anti-tumor activity, however, now it is recognized as a pleiotropic cytokine playing important roles in a host of biological processes and pathologies. To date, there are ten known members of the TNF-related cytokine family, TNF-alpha, TNF-beta (lymphotoxin-alpha), LT-beta, TRAIL and ligands for the Fas receptor, CD30, CD27, CD40, OX40 and 4-1BB receptors. These proteins have conserved C-terminal sequences and variable N-terminal sequences which are often used as membrane anchors, with the exception of INF-beta. Both TNF-alpha and TNF-beta function as homotrimers when they bind to TNF receptors.

TNF is produced by a number of cell types, including monocytes, fibroblasts, T-cells, natural killer (NK) cells and predominately by activated macrophages. TNF-alpha has been reported to have a role in the rapid necrosis of tumors, immununostimulation, autoimmune disease, graft rejection, producing an anti-viral response, septic shock, cerebral malaria, cytotoxicity, protection against deleterious effects of ionizing radiation produced during a course of chemotherapy, such as denaturation of enzymes, lipid peroxidation and DNA damage (Nata et al., J. Immunol. 136(7):2483 (1987)), growth regulation, vascular endothelium effects and metabolic effects. TNF-alpha also triggers endothelial cells to secrete various factors, including PAF-1, IL-1, GM-CSF and IL-6 to promote cell proliferation. In addition, TNF-alpha up-regulates various cell adhesion molecules such as E-Selectin, ICAM-1 and VCAM-1. TNF-alpha and the Fas ligand have also been shown to induce programmed cell death.

TNF-beta has many activities, including induction of an antiviral state and tumor necrosis, activation of polymorphonuclear leukocytes, induction of class I major histocompatibility complex antigens on endothelial cells, induction of adhesion molecules on endothelium and growth hormone stimulation (Ruddle, N. and Homer, R., Prog. Allergy 40:162-182 (1988)).

Both TNF-alpha and TNF-beta are involved in growth regulation and interact with hemopoietic cells at several stages of differentiation, inhibiting proliferation of various types of precursor cells, and inducing proliferation of immature myelomonocytic cells. Porter, A., Tibtech 9:158-162 (1991).

Recent studies with “knockout” mice have shown that mice deficient in TNF-beta production show abnormal development of the peripheral lymphoid organs and morphological changes in spleen architecture (reviewed in Aggarwal et al., Eur Cytokine Netw, 7(2):93-124 (1996)). With respect to the lymphoid organs, the popliteal, inguinal, para-aortic, mesenteric, axillary and cervical lymph nodes failed to develop in TNF-beta -/- mice. In addition, peripheral blood from TNF-beta -/- mice contained a three fold reduction in white blood cells as compared to normal mice. Peripheral blood from TNF-beta -/- mice, however, contained four fold more B cells as compared to their normal counterparts. Further, TNF-beta, in contrast to TNF-alpha has been shown to induce proliferation of EBV-infected B cells. These results indicate that TNF-beta is involved in lymphocyte development.

The first step in the induction of the various cellular responses mediated by TNF-alpha or TNF-beta is their binding to specific cell surface or soluble receptors. Two distinct TNF receptors of approximately 55-KDa (TNF-RI) and 75-KDa (TNF-RII) have been identified (Hohman et al., J. Biol. Chem., 264:14927-14934 (1989)), and human and mouse cDNAs corresponding to both receptor types have been isolated and characterized (Loetscher et al., Cell, 61:351 (1990)). Both TNF-Rs share the typical structure of cell surface receptors including extracellular, transmembrane and intracellular regions.

These molecules exist not only in cell bound forms, but also in soluble forms, consisting of the cleaved extra-cellular domains of the intact receptors (Nophar et al., EMBO Journal, 9 (10):3269-76 (1990)) and otherwise intact receptors wherein the transmembrane domain is lacking. The extracellular domains of TNF-RI and TNF-RII share 28% identity and are characterized by four repeated cysteine-rich motifs with significant intersubunit sequence homology. The majority of cell types and tissues appear to express both TNF receptors and both receptors are active in signal transduction, however, they are able to mediate distinct cellular responses. Further, TNF-RII was shown to exclusively mediate human T-cell proliferation by TNF as shown in PCT WO 94/09137.

TNF-RI dependent responses include accumulation of C-FOS, IL-6, and manganese superoxide dismutase mRNA, prostaglandin E2 synthesis, IL-2 receptor and MHC class I and II cell surface antigen expression, growth inhibition, and cytotoxicity. TNF-RI also triggers second messenger systems such as phospholipase A2, protein kinase C, phosphatidylcholine-specific phospholipase C and sphingomyelinase (Pfefferk et al., Cell, 73:457-467 (1993)).

Several interferons and other agents have been shown to regulate the expression of TNF receptors. Retinoic acid, for example, has been shown to induce the production of TNF receptors in some cells type while down regulating production in other cells. In addition, TNF-alpha has been shown to effect the localization of both types of receptor. TNF-alpha induces internalization of TNF-RI and secretion of TNF-RII (reviewed in Aggarwal et al., supra). Thus, the production and localization of both TNF-Rs are regulated by a variety of agents.

Both the yeast two hybrid system and co-precipitation and purification have been used to identify ligands which associate with both types of the TNF-Rs (reviewed in Aggarwal et al., supra and Vandenabeele et al., Trends in Cell Biol. 5:392-399 (1995)). Several proteins have been identified which interact with the cytoplasmic domain of a murine TNF-R. Two of these proteins appear to be related to the baculovirus inhibitor of apoptosis, suggesting a direct role for TNF-R in the regulation of programmed cell death.

The present invention relates to antibodies to a novel member of the Tumor Necrosis Factor (TNF) receptor family. More specifically, antibodies which specifically bind to a novel human TNF receptor-related protein, referred to herein as the TR2 receptor of SEQ ID NO: 2. (SEQ ID NO: 1 is cDNA sequence encoding TR2 receptor.)

Our studies have shown that TR2 participates in the interaction of T cells with antigen presenting cells, activation of T cells, induction of inflammatory mediators such as cytokines and induction of immunoglobulin production by B cells. We have shown that TR2 is involved in allogeneic proliferative responses which results from the interaction of T cells and antigen presenting cells such as B lymphocytes and monocytes/macrophages. Interactions of cells of the immune and hematopoietic system including T and B lymphocytes and cells of the myeloid lineage have been shown to be responsible for initiating and propagating pathological conditions such as inflammatory disorders, transplant rejections; autoimmune disorders, including but not limited to, systemic lupus erythomatosus (SLE); idiopathic thrombocytopenic purpura (ITP); rheumatoid arthritis (RA); multiple sclerosis (MS); psoriasis, inflammatory bowel disease (IBD); insulin dependent diabetes melititus (IDDM); allergic disorders, including asthma, allergic rhinitis, and atopic dermatitis; cancers, such as, lymphomas and leukemias; atherosclerosis; and viral infections, such as HSV infections and AIDS.

We have shown that TR2 is involved in IgE production and hence has potential utility in allergic disorders, including, asthma and allergic rhinitis. The production of IgE has been linked to mechanism of disease in atopic asthma and allergic disease. This disorder is characterised by the increased ability of B lymphocytes to produce IgE antibodies in response to allergens which are presented to the immune system via ingestion, inhalation or penetration through the skin. IgE binds to high affinity receptors on mast cells and basophils and in the presence of specific antigen, triggers the release of vasoactive mediators, chemoattractants and cytokines associated with the symptoms of type I allergic hypersensitivity. The interaction of IgE with low affinity receptors present on B lymphocytes and platelets and with both the high and low affinity receptors on monocytes and eosinophils contributes to the chronic inflammatory response seen in atopic individuals. In addition, there is a close association between levels of serum IgE and airway hyperresponsiveness, suggesting that in a significant number of patients, allergy facilitates the development of clinical asthma.

TR2 has recently been identified as a receptor utilised by HSV to enter cells. Antibodies to TR2 are expected to have utility in modulating HSV infection or immune responses to HSV.

Thus it is the intention of the present invention to provide a method for treating the above mentioned pathological conditions comprised of providing a patient with antibodies to the TR2 receptor. The antibodies of the invention include monoclonal antibodies (Mabs) and polyclonal antibodies.

### SUMMARY OF THE INVENTION

The present invention relates to antibodies, one prefered embodiment being monoclonal antibodies (mAbs) but also including polyclonal antibodies, to novel members of the Tumor Necrosis Factor (TNF) receptor family called TR2 receptor and their uses in pathological conditions.

Hybridoma cell lines producing such mAbs, methods of in vivo imaging of a pathological conditions, and methods of treating and diagnosing pathological conditions, caused by abnormal functioning, production or metabolism of TR2 receptors are also provided. In vitro assays for detecting the presence of TR2 and for evaluating the binding affinity of a test compound are also described.

### DETAILED DESCRIPTION OF THE PREFERRED EMBODIMENTS

One aspect of this invention relates to antibodies to TR2 receptor polypeptide. One preferred class of antibodies of the present invention is monoclonal antibody. The monoclonal antibody according to this invention includes any naturally or non-naturally occurring polypeptide which binds to an epitope on TR2 receptor, inhibits TR2 ligand binding with TR2 receptor, agonizes or antagonizes TR2 receptor. For example antibodies can be those having the binding specificity of 12C5, 18D4 or 3D6 (as described below). Preferably the monoclonal antibody or fragment thereof of the present invention binds to the linear or conformational epitope of the extra cellular domain of human TR2. Examples of such polypeptides include a half antibody molecule (a single heavy:light chain pair), or a fragment, such as the univalent fragments Fab or Fab′ and the divalent fragment F(ab)2 (“FAB” meaning fragment antigen binding) A fragment, according to the present invention may also be a single chain Fν fragment produced by methods well known in the art. See Skerra et al. Science 240: 1038-1041 (1988) and King et al. Biochemical J. 290: 723-729 (1991), each of which is hereby incorporated by reference. The monoclonal of the present invention also includes a non-peptide compound which is a “mimetic,” i.e. a compound that mimics the epitope binding site, resistant to proteolysis and non-immunogenic. Conformationally restricted cyclic organic peptides which mimic, for example, 12C5 or 18D4, can be produced in accordance with method well-known to the skilled artisan. See e.g., Saragovi, et al., Science 253:792-795 (1991), hereby incorporated by reference. The monoclonal antibody of the present invention also includes anti-idiotypic antibodies produced by methods well-known to the art of the invention. See e.g. Cozenza, Eur. J. Immonol. 6: 114 (1976) and Harlow et al., Antibodies: A Laboratory Manual, Cold Spring Harbor Publications pp. 726 (1988), each of which is hereby incorporated by reference.

The term “epitope” as used in describing this invention, includes any determinant responsible for the specific interaction with an antibody molecule. Epitopic determinants usually consist of chemically active surface groupings of molecules such as amino acids or sugar side chains and have specific three-dimensional structural characteristics.

The monoclonal antibody according to this invention also includes monoclonal antibody conjugates, which are for example, enzymes such as horseradish peroxidase, alkaline phosphatase, β-D-galactosidase, glucose oxidase, glucoamylase, carbanhydrase, acetyl-cholinesterase, lysozyme, malate dehydrogenase or glucose-6 phosphate dehydro. Fluorescent markers are also suitable conjugates and include fluorescein, fluorochrome, rhodamine, and the like. In such conjugates, the monoclonal antibody of the invention is bound to the enzymes or fluorescent markers directly or by way of a spacer or linker group, such as ethylenediaminetetraacetic acid (EDTA), diethylenetriaminepentaacetic acid (DPTA), or the like. Other conjugates include chemiluminescents such as luminal and imidizol and bioluminescents such as luciferase and luciferin. Cytostatics are also applicable as conjugates for the monoclonal antibody of the present invention and include alkylating substances, such as mechlorethamine, triethylene phosphoramide, triaziquone, camustine, semustine, methotrexate, mercaptopurine, cytarabine, fluorouracile, antibiotics such as actinomycine, and hormones and hormone antagonists such as corticosteroids, such a prednisone or progestins. The monoclonal antibody conjugates may be prepared by conjugating a cytotoxic substance containing either the intact toxin or the A-chain derived from it to the monoclonal antibody or fragment thereof, according to techniques well-known in the art. Chaudry et al. J. Biol. Chem. 268:9437-9441 (1993); Sung et al. Cancer Res. 53:2092-2099 (1993) and Selvaggi et al., J. Immuno-therapy 13:201-207 (1993), each of which is hereby incorporated by reference.

In one embodiment, the monoclonal antibody of the invention or fragment thereof is conjugated to a detectable label that is a radioisotope, such as ³H, ¹²⁵I, ¹³¹I, ³²P, ³⁵S, ¹⁴C, ⁵¹Cr, ³⁶Cl, ⁵⁷Co, ⁵⁸Co, ⁵⁹Fe, ⁷⁵Se, ¹⁵²Eu, and ^{99m}Tc which can be detected by known means such as gamma and scintillation counters, autoradiographs and the like.

The monoclonal antibody of the present invention may also be a monoclonal heteroconjugate, i.e., a hybrid of two or more antibody molecules. A suitable heteroconjugate includes, for instance, half of the 12C5 or 18D4 monoclonal antibody or fragment thereof and half of another monoclonal antibody, which is specific for a surface molecule on an immune effector cell, such as an NK cell or a macrophage. See Kerr et al. J. Immun. 144:4060-4067 (1990); Hsieh-Ma et al. Cancer Res. 52:6832-6839 (1992), hereby incorporated by reference.

In another embodiment, the monoclonal antibody of the invention is a chimeric monoclonal antibody. In one approach, the chimeric monoclonal antibody is engineered by cloning recombinant DNA containing the promoter, leader, and variable-region sequences from a mouse monoclonal, for example, 12C5 or 18D4, gene and the constant-region exons of a human antibody gene. The antibody encoded by such a recombinant gene is a mouse-human chimera. Its antibody specificity is determined by the variable region derived from mouse DNA; its isotype, which is determined by the constant region, is derived from human DNA. See Verhoeyn et al. BioEssays 8: 74 (1988), hereby incorporated by reference.

In another embodiment, the monoclonal antibody of the present invention is a “humanized” monoclonal antibody, produced by techniques well-known in the art. Carter et al., PNAS 89:4285-4289 (1992); Singer et al., J. Immun. 150:2844-2857 (1992) and Mountain et al. Biotechnol. Genet. Eng. Rev. 10:1-142 (1992), each of which is hereby incorporated by reference. That is, mouse complementary determining regions (“CDRs”) are transferred from heavy and light V-chains of the mouse Ig into a human V-domain, followed by the replacement of some human residues in the framework regions by their murine counterparts. “Humanized” monoclonal antibodies in accordance with this invention are suitable for use in in vivo diagnostic and therapeutic methods.

Monoclonal antibodies can be produced in various ways using techniques well-understood by those having ordinary skill in the art. Details of these techniques are described in Antibodies: A Laboratory Manual, Harlow et al. Cold Spring Harbor Publications, p. 726 (1988), which is hereby incorporated by reference.

Efforts to elicit immune responses and generate monoclonal antibodies without time consuming protein purification steps have taken advantage of rodent fibroblast transfectants expressing such proteins as v-fms or the CSF-1 receptor Sherr, et al., Blood 73: 1786-1793 (1989), hereby incorporated by reference.

The monoclonal antibodies and fragments thereof according to this invention are multiplied according to in vitro and in vivo methods well-known in the art. Multiplication in vitro is carried out in suitable culture media such as Dulbecco's Modified Eagle Medium or RPMI 1640 medium, optionally replenished by a mammalian serum such as fetal calf serum or trace elements and growth-sustaining supplements, e.g. feeder cells, such as nonnal mouse peritoneal exudate cells, spleen cells, bone marrow macrophages or the like. In vitro production provides relatively pure antibody preparations and allows scale-up to give large amounts of the desired antibodies. Techniques for large scale hybridoma cultivation under tissue culture conditions are known in the art and include homogenous suspension culture, e.g., in an airlift reactor or in a continuous stirrer reactor or immobilized or entrapped cell culture.

Large amounts of the monoclonal antibody of the present invention may also be obtained by multiplying hybridoma cells in vivo. Cell clones are injected into mammals which are histocompatible with the parent cells, e.g. syngeneic mice, to cause growth of antibody-producing tumors. Optionally, the animals are primed with a hydrocarbon, especially oils such as pristane (tetramethyl-pentadecane) prior to injection. After one to three weeks, the desired monoclonal antibody is recovered from the body fluid of the mammal.

In accordance with the present invention, fragments of the monoclonal antibody of the invention can be obtained from the monoclonal antibody produced as described above, by methods which include digestion with enzymes such as pepsin or papain andlor cleavage of disulfide bonds by chemical reduction. Alternatively, monoclonal antibody fragments encompassed by the present invention can be synthesized using an automated peptide synthesizer as supplied by Applied Biosystems, Multiple Peptide Systems, etc., or they may be produced manually, using techniques well known in the art. See Geysen, et at. J. Immunol. Methods 102:259-274 (1978), hereby incorporated by reference.

The monoclonal conjugates of the present invention are prepared by methods known in the art, e.g., by reacting a monoclonal antibody prepared as described above with, for instance, an enzyme in the presence of a coupling agent such as glutaraldehyde or periodate. Conjugates with fluorescein markers are prepared in the presence of these coupling agents or by reaction with an isothiocyanate. Conjugates with metal chelates are similarly produced.

Radioactively labeled monoclonal antibodies of the present invention are produced according to a well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium-99 m by ligand exchange process, for example, by reducing pertechnate with stannous solution, chelating the reduced technetium onto a Sephadex column and applying the antibody to this column or by direct labelling techniques, e.g. by incubating pertechnate, a reducing agent such as SNC12, a buffer solution such as sodium-potassium phthalate solution, and the antibody.

Thus, in one embodiment, the invention relates to a pharmaceutical composition for in vivo imaging of a pathological condition that expresses TR2 receptors comprising the monoclonal antibody or fragment thereof of the invention which binds TR2 receptor in vivo; and a pharmaceutically acceptable carrier.

In another embodiment, the invention relates to an in vivo method of imaging a pathological condition which expresses TR2 receptors using the above pharmaceutical composition.

Thus, with the discovery of monoclonal antibodies including, but not limited to 12C5 or 18D4, the applicants have also discovered an in vivo method of imaging a pathological condition caused by abnormal functioning, production or metabolism of TR2 receptors. Specifically, this method involves administering to a subject an imaging-effective amount of a detectably labeled monoclonal antibody or fragment thereof, and a pharmaceutically effective carrier; and detecting the binding of the labeled monoclonal antibody to the TR2 receptors in the pathological condition.

The term “pathological condition” refers to an abnormallity or disease, as these terms are commonly used. The pathological conditions of the present invention are those which are brought about by improper or inappropriate expression, production or metabolism of TR2 receptors, such as, inflammatory disorders; transplant rejections; autoimmune disorders, including but not limited to, systemic lupus erythomatosus (SLE); idiopathic thrombocytopenic purpura (ITP); rheumatoid arthritis (RA); multiple sclerosis (MS); psoriasis, inflammatory bowel disease (IBD); insulin dependent diabetes melititus (IDDM); allergic disorders, including asthma, allergic rhinitis, and atopic dermatitis; cancers, such as, lymphomas and leukemias; atherosclerosis; and viral infections, such as HSV infections and AIDS.

The term “in vivo imaging” refers to any method which permits the detection of a labeled monoclonal antibody of the present invention or fragment thereof that specifically binds to the TR2 located in the subject's body. A “subject” is a mammal, preferably a human, and most preferably a human known to have a neoplasia that expresses TR2 receptors.

An “imaging effective amount” means that the amount of the detectably labeled monoclonal antibody or fragment thereof administered is sufficient to enable detection of binding of the monoclonal antibody or fragment thereof to the TR2 receptor.

Generally, the dosage of the detectably labeled monoclonal antibody or fragment thereof will vary depending on consideration such as age, condition, sex, and extent of disease in the patient, counter-indications, if any, concomitant therapies and other variables, to be adjusted by a physician skilled in the art. Dosage can vary from 0.01 mg/kg to 2,000 mg/kg, preferably 0.1 mg/kg to 1,000 mg/kg.

As noted above, the present invention encompasses monoclonal antibody conjugates in which the conjugate may be a detectable label. For purposes of in vivo imaging, the type of detection instrument available is a major factor in selecting a given label. For instance, radioactive isotopes and paramagnetic isotopes are particularly suitable for in vivo imaging in the methods of the present invention. The type of instrument is used will guide the selection of the radionuclide. For instance, the radionuclide chosen must have a type of decay which is detectable for a given type of instrument. However, any conventional method for visualizing diagnostic imaging can be utilized in accordance with this invention.

Another factor to consider in selecting a radionuclide for in vivo diagnosis is that the half-life of a nuclide be long enough so that it is still detectable at the time of maximum uptake by the target, but short enough so that deleterious radiation upon the host, as well as background, is minimized. Ideally, a radionuclide used for in vivo imaging will lack a particulate emission, but produce a large number of photons in a 140-2000 keV range, which may be readily detected by conventional gamma cameras.

As discussed above in connection with the production of monoclonal conjugates, a radionuclide may be bound to an antibody either directly or indirectly by using an intermediary functional group. Intermediary functional groups which are used to bind radioisotopes which exist as metallic ions to antibody are diethylenetriaminepentaacetic acid (DTPA) and ethylene diaminetetracetic acid (EDTA). Examples of metallic ions suitable for use in this invention are ^{99m}Tc, ¹²³I, ¹³¹I, ¹¹¹In, ¹³¹I, ⁹⁷Ru, ⁶⁷Ga, ¹²⁵I, ⁶⁸Ga, ⁷²As, ⁸⁹Zr, and ²⁰¹Tl.

In accordance with this invention, the monoclonal antibody or fragment thereof may be labeled by any of several techniques known to the art. See, e.g., Wagner et al., J. Nucl. Med. 20:428 (1979) and Saha et al., J. Nucl. Med. 6:542 (1976), hereby incorporated by reference.

The methods of the present invention may also use pararnagnetic isotopes for purposes of in vivo detection. Elements particularly useful in Magnetic Resonance Imaging (“MRI”) include ¹⁵⁷Gd, ⁵⁵Mn, ¹⁶²Dy, ⁵²Cr, and ⁵⁶Fe.

Administration to the subject may be local or systemic and accomplished intravenously, intraarterially, via the spinal fluid or the like. Administration may also be intradermal or intracavitary, depending upon the body site under examination. After a sufficient time has lapsed for the monoclonal antibody or fragment thereof to bind with the TR2, for example 30 minutes to 48 hours, the area of the subject under investigation is examined by routine imaging techniques such as MRI, SPECT, planar scintillation imaging and emerging imaging techniques, as well. The exact protocol will necessarily vary depending upon factors specific to the patient, as noted above, and depending upon the body site under examination, method of administration and type of label used; the determination of specific procedures would be routine to the skilled artisan. The distribution of the bound radioactive isotope and its decrease with time is then monitored and recorded. By comparing the results with data obtained from studies of clinically normal individuals, the presence and location of the abnormality can be determined and monitored.

The pharmaceutical composition of the present invention are advantageously administered in the form of injectable compositions. And in some instances they may also be administered by inhalation. A typical composition for such purpose comprises a pharmaceutically acceptable carrier. For instance, the composition may contain about 10 mg of human serum albumin and from about 20 to 200 micrograms of the labeled monoclonal antibody or fragment thereof per milliliter of phosphate buffer containing NaCl. Other pharmaceutically acceptable carriers include aqueous solutions, non-toxic excipients, including salts, preservatives, buffers and the like, as described, for instance, in Remington's Pharmaceutical Sciences, 15th Ed. Easton: Mack Publishing Co. pp 1405-1412 and 1461-1487 (1975) and The National Formulary XIV, 14th Ed. Washington: American Pharmaceutical Association (1975), the contents of which are hereby incorporated by reference. Examples of non-aqueous solvents are propylene glycol, polyethylene glycol, vegetable oil and injectable organic esters such as ethyloeate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial, anti-oxidants, chelating agents and inert gases. The pH and exact concentration of the various components the pharmaceutical composition are adjusted according to routine skills in the art. See Goodman and Gilman's The Pharmacological Basis for Therapeutics (7th Ed.).

Particularly preferred pharmaceutical compositions of the present invention are those that, in addition to specifically binding the TR2 in vivo, are also non-toxic at appropriate dosage levels and have a satisfactory duration of effect.

In another embodiment, the invention relates to a method using the antibodies of the present invention to inhibiting or treating pathological conditions such as: inflammatory disorders; transplant rejections; autoimmune disorders, including but not limited to, systemic lupus erythornatosus (SLE); idiopathic thrombocytopenic purpura (ITP); rheumatoid arthritis (RA); multiple sclerosis (MS); psoriasis, inflammatory bowel disease (IBD); insulin dependent diabetes melititus (IDDM); allergic disorders, including asthma, allergic rhinitis, and atopic dermatitis; cancers, such as, lymphomas and leukemias; atherosclerosis; and viral infections, such as HSV infections and AIDS. More specifically, the invention relates to a method of inhibiting or treating the above mentioned pathological conditions with a therapeutic amount of a monoclonal antibody or fragment thereof which specifically binds to an epitope of TR2 receptor, inhibits TR2 ligand binding to TR2 receptor, or antagonizes or agonizes TR2, by administering to a patient in need thereof a therapeutic amount of the antibody of the present invention. Alternatively, a patient with the above pathological conditions can be treated with the antibodies by taking cells or tissues and incubating them with therapeutic amounts of antibodies ex-vivo; and re-administering the cells or tissues back to the patient.

The quantity of antibody of the present invention necessary for effective therapy will depend upon many different factors, including the means of administration, target site, physiological state of the patient, other medicants administered, etc. Thus treatment dosages should be titrated to optimize safety and efficacy. Typically, dosages used in vitro may provide useful guidance in the amounts useful for in situ administration of the monoclonal antibody, and as noted above, animal models may be used to determine effective dosages for treatment of particular disorders. Various considerations are described, e.g. in Gilman et al. (eds.)(1990), Goodman and Gilman's: The Pharmacological Bases of Therapeutics, 8th Ed., Pergamon Press; and Remington's Pharmaceutical Sciences, 17th Ed. (1990), Mack Publishing Co., Easton, Pa., each of which is herein incorporated by reference. Methods of administration are discussed therein and include, oral, intravenous, intraperitoneal, or intra muscular administration, transdermal diffusion and others. Pharmaceutically acceptable carriers include, water, saline, buffers, and other compounds described, e.g., in Merck Index, Merck & Co., Rahway, N.J. Because of the high affinity of binding of the monoclonal antibody of the present invention with TR2, low dosages of pharmaceutical compositions for purposes of inhibiting neoplasia growth would be effective. Thus, dosage ranges would ordinarily be expected to be in amounts lower than 1 nM concentrations, typically less than 10 μM, usually less than about 100 nM, preferably less than about 10 nM (nanomolar), and most preferably less than about 1 fM (femtomolar), with the appropriate carrier. Slow release formulations, or slow release apparatus may be utilized for continuous administration.

In another embodiment, the invention relates to an in vitro assay for the detection of TR2 or its ligand in whatever kind of “sample” it may occur, such samples including fluid, semi-fluid or tissue samples, using the monoclonal antibody or fragment thereof of the invention. The assay can be a competitive or sandwich assay, or any assay well-known to the artisan which depends on the formation of an antibody-antigen immune complex. For purposes of this invention, the monoclonal antibody or fragment thereof can be immobilized or labeled. Many carriers are known to be the skilled artisan to which the monoclonal, antibody or fragments thereof of the present invention can be bound for immobilization. Where required, derivatization techniques can be used for immobilizing the monoclonal antibody or fragment thereof on a substrate. Well-known carriers include glass, polystyrene, polypropylene, polyethylene, dextran, nylon, amylases, natural and modified celluloses etc. The carrier can be either soluble or insoluble. Immunoassays encompassed by the present invention include, but are not limited to those described in U.S. Pat. Nos. 4,367,110 (double monoclonal antibody sandwich assay); Wide et al., Kirkham and Hunter, eds. Radioimmunoassay Methods, E. and S. L:ivingstone, Edinburgh (1970); U.S. Pat. No. 4,452,901 (western blot); Brown et al., J. Biol. Chem. 255:4980-4983 (1980) (immunoprecipitation of labeled ligand); and Brooks et al., Clin. Exp. Immunol. 39:477 (1980) (immunocytochemistry).

The monoclonal antibodies and fragments thereof of the present invention may be used in in vitro assays designed to screen compounds which bind to TR2 (including agonists and antagonists) or its ligand. See Fodor et al. Science 251: 767-773 (1991), incorporated herein by reference. A method of using antibodies to TR2 for screening of compounds which agonizes or antagonizes TR2 comprise detecting the alteration of TR2 activity level in the presence of both TR2 antibodies and a candidate molecule which might otherwise be occupied by TR2 receptor ligand. Thus, the present invention contemplates a competitive drug screening assay, where the monoclonal antibodies or fragments thereof of the invention compete with a test compound for binding to TR2. In this manner the monoclonal antibodies and fragments thereof are used to detect the presence of any polypeptide or molecule which shares one or more binding sites of the TR2 and can be used to occupy binding sites on the receptor which might otherwise be occupied by TR2 receptor ligand.

In vitro assays in accordance with the present invention also include the use of isolated membranes from cells expressing a recombinant TR2 receptor, or fragments attached to solid phase substrates. These assays allow for the diagnostic determination of the effects of either binding segment mutations and modifications, or ligand mutations and modifications, e.g., ligand analogues.

The monoclonal antibodies of the present invention are suitable for use in a kit. Such a kit may comprise a receptacle being compartmentalized to receive one or more containers such as vials, tubes and the like, such containers holding separate elements of the invention. For example, one container may contain a first antibody bound to an insoluble or partly soluble carrier. A second container may contain soluble, detectably-labeled second antibody, in lyophilized form or in solution. The receptacle may also contain a third container holding a detectably labeled third antibody in lyophilized form or in solution. A kit of this nature can be used in the sandwich assay of the invention.

Without further elaboration, it is believed that one skilled in the art can, using the preceding description, utilize the present invention to its fullest extent. The following examples are merely illustrative and not limitative of the remainder of the disclosure in any way whatsoever.

### EXAMPLES

The examples below are carried out using standard techniques, which are well known and routine to those of skill in the art, except where otherwise described in detail. The examples illustrate, but do not limit the invention.

### Generation of TR2-Ig

### Expression and Purification of TR2-Fc(TR2-Ig Fusion Protein) and Cleaved TR2

The putative transmembrane domain of translated TR2 receptor was determined by hydrophobicity using the method of Goldman et al. (Ann. Rev. of Biophys. Biophys. Chem. 15:321-353 (1986)) for identifying nonpolar transbilayer helices. The region upstream of this transmembrane domain, encoding the putative leader peptide and extracellular domain, was chosen for the production of an Fc fusion protein. Primers were designed to PCR the corresponding coding region from HTXBS40 (a clone containing TR2 receptor clone) with the addition of a BglII site (single underlined), a Factor Xa protease site and an Asp718I site (double underlined) at the 3 end. PCR with this primer pair (forward 35-mer: 5′ CAGGAATTCGCAGCCATGGAGCCTCCTGGAGACTG 3′ (SEQ ID NO:3), and reverse primer 53-mer: 5′ CCATACCCAGGTACCCCTTCCCTCGATAGATCT TGCCTTCGTCACCAGCCAGC 3′ (SEQ ID NO:4)), which contains 18 nucleotides of the TR2 coding sequence, resulted in one band of the expected size. This was cloned into COSFclink to give the TR2-Fclink plasmid. The PCR product was digested with EcoRI and Asp718I and ligated into the COSFclink plasmid (Johansen, et al., J. Biol. Chem. 270:9459-9471 (1995)) to produce TR2-Fclink.

COS cells were transiently transfected with TR2-Fclink and the resulting supernatant was immunoprecipitated with protein A agarose. Western blot analysis of the immunoprecipitate using goat anti-human Fc antibodies revealed a strong band consistent with the expected size for glycosylated TR2-Fc (greater than 47.5 kD). A 15 L transient COS transfection was performed and the resulting supernatant was purified (see below). The purified protein was used to immunize mice following DNA injection for the production of mAbs.

CHO cells were transfected with TR2-Fclink to produce stable cell lines. Five lines were chosen by dot blot analysis for expansion and were adapted to shaker flasks. The line with the highest level of TR2-Fc protein expression was identified by Western blot analysis. TR2-Fc protein purified from the supernatant of this line was used for cell binding studies by flow cytometry, either as intact protein or after factor Xa cleavage and biotinylation.

Clone HTXBS40 is an allelic variant of TR2 which differs from the sequence shown in SEQ ID NO: 1 in that HTXBS40 contains guanine at nucleotide 314, thymine at nucleotide 386 and cytosine at nucleotide 627.

A plasmid suitable for expression of the extracellular domain of TR2 was constructed as follows to immunize mice for the production of anti-TR2 mAbs. The Fc fragment was removed from TR2-Fclink by a BglII/XbaI digestion, Klenow was used to fill in the overhangs, and the blunt ends of the plasmid were religated. The resulting frame shift introduced a stop codon immediately following the amino acids which bad originally been introduced into TR2-Fclink by the addition of the BglII site. Thus, the C terminus of the extracellular domain of TR2 is followed by only 2 amino acids (RS) in this constructed (TR2exlink).

### Purification of TR2-Fc from CHO ElA Conditioned Media Followed by Cleavage and Biotinylation of TR2.

Assays

Product purity through the purification was monitored on 15% Laemmli SDS-PAGE gels run under reducing and non-reducing conditions. Protein concentration was monitored by A280 assuming an extinction coefficient of 0.7 for the receptor and 1.28 for the chimera, both calculated from the sequence. Extinction coefficients were confirmed by AAA.

Protein G Chromatography of the TR2-Fc Fusion Protein

All steps described below were carried out at 4° C. 15 L of CHO conditioned media (CM) (0.2 μ filtered following harvest in cell culture) was applied to a 5×10 cm column of Protein G at a linear flow rate of 199 cm/h. The column had been washed with 100 mM glycine, pH 2.5 and equilibrated in 20 mM sodium phosphate, 150 mM sodium chloride, pH 7 prior to sample application. After the CM was loaded the column was washed with 5 column volumes of 20 mM sodium phosphate, 150 mM sodium chloride, pH 7 and eluted with 100 mM glycine, pH 2.5. 435 ml of eluate was immediately neutralized with 3 M Tris, pH 8.5 and 0.2 μ filtered. Based on A280, extinction coefficient 1.28, 65 mg of protein was recovered at 0.15 mg/ml.

Concentration/Dialysis

385 ml of Protein G eluate was concentrated in an Amicon stirred cell fitted with a 30K membrane to 34 nl at a final concentration of 1.7. The concentrate was dialyzed against buffer.

Factor Xa Cleavage and Purification to Generate Free Receptor

Six ml (10.2 mg) of TR2-Fc was added to 50 μg of Factor Xa resulting in a 1:200, e:s ratio. The mixture was incubated overnight at 4° C.

Protein G Chromatography of the Free TR2 receptor

A 1 ml column of Protein G was equilibrated in 20 mM sodium phosphate, 150 mM sodium chloride, pH 6.5 in a disposable column using gravity flow. The cleaved receptor was passed over the column 3 times after which the column was washed with 20 mM sodium phosphate, 150 mM sodium chloride, pH 6.5 until no A280 absorbance was seen. The column was eluted with 2.5 ml of 100 mM glycine, pH 2.5 neutralized with 83 el of 3 M Tris, pH 8.5. TR2 eluted in the nonbound fraction.

Concentration

The nonbound fraction from the Protein G column, about 12 ml, was concentrated in a Centricon 10K cell (Amicon) to about 1 ml to a final concentration of 3.5 mg/ml estimated by A280, extinction coefficient 0.7.

Mono S Chromatography

The concentrated sample was diluted to 5 ml with 20 mM sodium phosphate, pH 6 and applied to a 0.5×5 cm Mono S column equilibrated in 20 mM sodium phosphate, pH 6 at a linear flow rate of 300 cm/h. The column was washed with 20 mM sodium phosphate, pH 6 and eluted with a 20 column volume linear gradient of 20 mM sodium phosphate, pH 6 to 20 mM sodium phosphate, 1 M sodium chloride, pH 6. TR2 protein eluted in the nonbound fraction.

Concentration/Dialysis

The 3 ml nonbound fraction from the Mono S column was concentrated to 1 ml as above using a Centricon 10K cell and dialyze against 20 mM sodium phosphate, 150 mM sodium chloride, pH 7. The concentration following dialysis was 2.1 mg/ml.

Biotinylation

0.5 mg of TR2 at 2.1 mg/ml was dialyzed against 100 mM borate, pH 8.5. A 20-fold molar excess of NHS-LC Biotin was added and the mixture was left on a rotator overnight at 4øC. The biotinylated TR2 was dialyzed against. 20 mM sodium phosphate, 150 mM sodium chloride, pH 7, sterile filtered and stored at −70øC. Biotinylation was demonstrated on a Western blot probed with strepavidin HRP and subsequently developed with ECL reagent.

Monoclonal antibody generation

Mice (F1 hybrids of Balb/c and C57BL/6) were immunised subcutaneously with 10 ug recombinant TR2 in Freunds complete adjuvant and 4 weeks later with 10 ug TR2 in Freunds incomplete adjuvant. On the basis of a good serum antibody titre to TR2 one mouse received further immunisations of 8 ug TR2 (i.p. in saline) at 8 weeks, and two days later. Two days following the final immunisation a splenectomy was performed. Mouse spleen cells were used to prepare hybridomas by standard procedures, (Zola, H.Ed., Monoclonal Antibodies, CRC Press Inc. 1987). Positive hybridomas were cloned by the limiting dilution method.

Hybridoma Screening Assay

96-well plates were coated with TR2-Fc (0.25 ug/ml, 100 ul/well in PBS) by incubation overnight at 4° C. The solution was then aspirated and non-specific binding sites were blocked with 250 ul/well of 1% bovine serum albumin (BSA) in TBS buffer (50 mM Tris, 150 mM NaCl, 0.02% Kathon, pH 7.4) for 5-60 minutes at RT. Following this and each of the following steps, the plate was washed 4 times in wash buffer (10 mM Tris, 150 mM NaCl, 0.05% Tween 20, 0.02% Kathon, pH 7.4). To each well, 50 uL hybridoma medium and 50 uL assay buffer (0.5% BSA, 0.05% bovine gamma globulin, 0.01% Tween 40, 20 uM diethylenetriaminepentaacetic in TBS buffer) was added and the plates were incubated for 60 min at RT in a shaker-incubator, followed by an incubation of 60 min at RT in a shaker-incubator with 100 ul 0.5 ug/ml Eu³⁺ labelled anti-mouse antibody in assay buffer. Finally 100 ul /well of enhancer (Wallac) was added and incubated for 5 min at RT and the fluorescence measured. Hybridomas having counts >100K were expanded into 24-well plates.

Immunoassay

To determine the specificity of the anti-TR2 Mabs generated 96-well plates were coated (1 ug/ml TR2-Fc, 100 ul/well) and blocked as above with TR2-Fc. All the following incubations were performed in a shaker-incubator at RT. After washing the wells 50 ul TR2 (2 ug/ml), TR2-Fc (2 ug/ml), hIgG (2 ug/ml) or assay buffer and 50 ul Mab were added and incubated for 60 min. After washing the wells 100 ul 0.5 ug/ml Eu³⁺ labelled anti-mouse antibody in assay buffer was added for 60 min, the wells washed and then 100 ul /well of enhancer (Wallac) was added and incubated for 5 min at RT and the fluorescence measured. All positive hybridomas showed displacement of binding with TR2 and TR2-Fc and none with hIgG.

Purification of Mabs

Mabs were purified by ProsepA (Bio Processing) chromatography respectively using the manufacturer's instructions. Mabs were >95% pure by SDS-PAGE.

Biosensor Studies

Surface plasmon resonance (SPR) technology (BIAcore) was used to analyse the epitope specificity and affinity of the TR2 Mabs.

18D4 and 12C5 have simnilar/overlapping epitopes

### Summary of Binding Data

### Epitope Determination of TR2 mAbs and Affinity Measurements by Surface Plasmon Resonance (SPR) Measurements Using a BIAcore Device

| mAb | Kₐₛₛ × 10⁻⁵ (M⁻¹s⁻¹) | K_{diss} × 10⁴ (s⁻¹) | calc. K_{D} (nM) |
|---|---|---|---|
| 12C5 | 5.9 | 5.4 | 0.91 |
| 18D4 | 0.57 | 10 | 18 |

The TR2-Fc was immobilized onto the sensor surface. Solutions of the mAbs were passed over the surface. Equilibrium responses at each mAb concentration were calculated from the kinetic data (see attachment). The different maximal responses of the mAbs suggest they bind to different epitopes. The “affinities” of the mAbs appear good for the TR2-Fc but may be considerably lower for an expressed monomeric receptor. The association rate constant is lower than usually seen for most mAbs. Maybe this is a clue.

### Binding of TR2 mAb 18D4 to Activated Human CD4⁺T Cells

Monoclonal antibodies 18D4 were tested for reactivity on freshly isolated activated CD4⁺T cells. CD4+ T cells were purified from human peripheral blood by ficoll density gradient centrifugation, the depletion of B lymphocytes and monocyte/macrophages in T cell columns (R&D Systems) and subsequent depletion of CD8⁺ T cells using immunomagnetic CD8 dynabeads (Dynal). Cells were stimulated with PHA (5 ug/ml) and PMA (10 ng/ml) for 72 hours in RPMI 1640 medium supplemented with 10% fetal calf serum, 2 mM L-Glutarnine, 50 ug/ml Gentomycin and 25 mM Hepes buffer. Activated cells were incubated with different concentrations of TR2 mAb 18D4for 30 minutes at 4° C., washed twice in PBS containing 0.2% BSA and 0.1% Sodium Azide (Staining buffer) and incubated for another 30 minutes at 4° C. with Goat anti-mouse FITC labelled antibody. Cells were washed three times and fixed in staining buffer containing 2% Formaldehyde. Samples were subsequently analysed on a Becton Dickinson FACSort using Cellquest software.

Specific binding to 72 hour activated CD4⁺T cells was demonstrated for the TR2 mAb. Optimal binding was seen at 100 ug/ml for 18D4. This data indicates that TR2 is expressed on the surface of activated CD4⁺T cells and that the TR2 mAb 18D4 binds to this molecule.

### Kinetics of TR2 Expression on Activated CD4⁺T Cells

Human peripheral blood CD4⁺T cells were isolated using density gradient centrifugation, T cell columns (R&D Systems) and depletion of CD8⁺T cells using immunomagnetic CD8 beads (Dynal). Cells were activated using immobilised anti-human CD3 mAb (1 ug/ml) in RPMI 1640 medium supplemented with 10% fetal calf serum, 2 mM L-Glutamine, 10 ug/ml Gentomycin and 25 mM Hepes buffer. At 24 hour intervals activated cells were incubated with TR2 mAbs for 30 minutes on ice, washed twice in PBS containing 0.2% BSA and 0.1% Sodium Azide (Staining buffer) and incubated for another 30 minutes with Goat anti-mouse FITC labelled antibody. Cells were washed three times and fixed in staining buffer containing 2% Formaldehyde. Samples were subsequently analysed on a Becton Dickinson FACSort using Cellquest software.

TR2 mAb 18D4 showed moderate levels of binding to resting CD4⁺T lymphocytes but after 24 hours of stimulation with immobilised anti-CD3 mAb 18D4 binding decreased to low levels. After 48 hours, levels of TR2 cell surface expression increased to maximal levels before declining slightly at 72 hours. This data suggests that TR2 is expressed on resting CD4⁺T cells and following anti-CD3 stimulation cell surface expression of TR2 increases to maximal levels by 48 hours.

### Inhibition of Mixed Lymphocvte Proliferation by TR2 mabs

Peripheral blood T cells express TR2 and the role of this receptor in T cell activation was examined using a mixed lymphocyte reaction (MLR) proliferation assay. Peripheral blood mononuclear cells (PBMCs) from three healthy donors were purified by density gradient centrifugation. PBMCs from two donors were adjusted to 1×10⁶ cells/ml in RPMI 1640 medium supplemented with 10% fetal calf serum, 2 mM L-Glutamine, 50 ug/ml Gentomycin and 25 mM Hepes buffer. PBMCs from the third donor were adjusted to 2×10⁵ cells/ml. Fifty microliters of PBMCs from each donor were added to wells of a 96 well round bottomed microtitre plate. Dilutions of TR2 mAbs 12C5 and 18D4, anti-human CD4 mAb and control anti-hIL-5 mAb 2B6 were added in quadruplicate to the plate. Cells were cultured for 6 days at 37° C. in 5% CO₂ and 1uCi of ³H thymidine was added to wells for the last 6 hours of culture. Cells were harvested using a Skatron cell harvester and ³H thymidine incorporation was determined using a Wallac 3 scintillation counter. Positive control anti-CD4 mAb inhibited MLR proliferation at all concentrations tested (0.5-100 ug/ml) whereas negative control mAb 2B6 failed to inhibit allogeneic proliferation. TR2 mAb 12C5 inhibited allogenic proliferation from 0.4-100 ug/ml. In comparison, TR2 mAb 18D4 inhibited proliferation from only 25-100 ug/ml. A primary component of MLR proliferation can be attributed to T cells as shown by inhibition with the anti-CD4 mAb. This data suggests that TR2 mAbs 12C5 and 18D4 inhibit allogenic proliferation responses and indicates that TR2 is involved in T cell activation.

### TR2 mAbs Inhibit Anti-CD3-Stimulated CD4±T Cell Proliferation and TNF Alpha Production

The capacity of TR2 Mabs to interfere with anti-CD3 driven CD4⁺T cell proliferation was examined. In addition, secreted TNFa levels were also determined. Human peripheral blood CD4⁺T cells were isolated by density gradient centrifugation, T cell columns and depletion of CD8⁺ T cells using magnetic CD8 dynabeads. Purified CD4⁺T cells were adjusted to 1×10⁶ cell/ml in RPMI 1640 medium supplemented with 10% fetal calf serum, 2 mM L-Glutamine, 50 ug/ml Gentomycin and 25 mM Hepes buffer. 96 well flat bottomed microtitre plates with immobilised anti-CD3 mAb (5 ug/ml) received 10 ul of cell suspension, 50 ul of either TR2 mab 12C5 or 18D4 dilutions and 50 ul of medium in quadruplicate. Cells were incubated at 37° C. in 5% CO₂. After 48 hours, 100 ul of supernatant was removed and pooled for each quadruplicate. 100 ul of fresh medium was then added back to each well.

Cells were cultured for another 24 hours and 1 uCi of ³H thymidine was added to wells for the last 6 hours of culture. Cells were harvested using a Skatron cell harvester and thymidine incorporation was determined using a Wallac β scintillation counter. Supernatant TNFa levels were determined using ELISA detection kit for human TNFa (R&D Systems)

Both TR2 mAbs 12C5 and 18D4 inhibited anti-CD3 induced CD4⁺T proliferation. 18D4 inhibited proliferation from 0.025-100 ug/ml whereas 12C5 showed activity from 0.0062-100 ug/ml suggesting that 12C5 was more active than 18D4. Complete inhibition of proliferation was seen with both mabs between 25 and 100 ug/ml. Mabs 18D4 and 12C5 inhibited CD4⁺T cell proliferation with IC₅₀'s of 8 nM and 0.5 nM, respectively. TNFa levels in culture supernatants followed a similar pattern, with 12C5 and 18D4 showing a dose dependent inhibition of TNFa production. 18D4 appeared to be more active than 12C5 at inhibiting TNFa production. Both TR2 mAbs completely suppressed TNFa production at 100 ug/ml.

This data suggests that TR2 mAbs 12C5 and 18D4 are capable of inhibiting anti-CD3-stimulated CD4⁺T cell proliferation and the production of TNFa indicating that TR2 is involved in modulating T cell proliferative responses and the production of T cell derived pro-inflammatory cytokines.

### TR2 mAbs Inhibit Anti-CD3 and Anti-CD28-Stimulated CD4⁺T Cell Proliferation. TNFa and IL-2 Production

The capacity of TR2 Mabs to interfere with anti-CD3 and anti-CD28 driven CD4⁺T cell proliferation was examined. In addition, the effect of TR2 Mabs on secreted TNFa and IL-2 levels were also determined. Human peripheral blood CD4⁺T cells were isolated by denisty gradient centrifugation, T cell columns and depletion of CD8⁺T cells using magnetic CD8 dynabeads. Purified CD4⁺T cells were adjusted to 1×10⁶ cell/ml in RPMI 1640 medium supplemented with 10% fetal calf serum, 2 mM L-Glutamine, 50 ug/ml Gentomycin and 25 mM Hepes buffer. 96 well flat bottomed microtitre plates with immobilised anti-CD3 mAb (5 ug/ml) received 100 ul of cell suspension, 50 ul of either TR2 mab 12C5 or 18D4 dilutions and 50 ul of anti-CD28 mAb in quadruplicate. Cells were incubated at 37° C. in 5% CO₂. After 48 hours, 100 ul of supernatant was removed and pooled for each quadruplicate. 100 ul of fresh medium was then added back to each well.

Cells were cultured for another 24 hours and 1 uCi of ³H thymidine was added to wells for the last 6 hours of culture. Cells were harvested using a Skatron cell harvester and thymidine incorporation was determined using a Wallac β scintillation counter. Supernatant TNFa and IL-2 levels were determined using ELISA detection kits for human TNFa and IL-2 (R&D Systems).

Both TR2 mAbs 12C5 and 18D4 inhibited anti-CD3 and CD28 mAb induced CD4⁺T proliferation. 18D4 inhibited proliferation from 1.5-100 ug/ml whereas 12C5 only showed activity from 25-100 ug/ml. Complete inhibition of proliferation was seen with both mabs at 100 ug/ml. Mabs 1 8D4 and 12C5 inhibited CD3/CD28 stimulated proliferation with IC ₅₀'s of 93 and 780 nM, respectively. TNFa levels in culture supernatants followed a similar pattern, with 12C5 and 18D4 showing a dose dependent inhibition of TNFa production. 18D4 appeared to be more active than 12C5 which correlated with the capacity of these mAbs to inhibit proliferation.

A similar dose dependent inhibition of IL-2 production by both TR2 mAbs was also observed, with no detectable IL-2 present in cells treated with 100 ug/ml TR2 mAb.

This data suggests that TR2 mAbs 12C5 and 18D4 are capable of inhibiting CD4⁺T cell proliferation and the production of cytokines such as TNFa and IL-2. This indicates that TR2 is involved in T cell proliferative responses, pro-inflammatory cytokine production and mitogenic T cell cytokine production.

### TR2 mAbs Inhibit in vitro IgE Production in Response to IL-4 and Anti-CD40 mAb

The capacity of TR2 mAb 12C5 to inhibit human IgE production was examined. Human peripheral blood mononuclear cell (PBMCs) were purified by density gradient centrifugation and adjusted to 1.25×10⁶ cells/ml in HB 101 medium supplemented with insulin (5 ug/ml), transferrin (5 ug/ml) and selenious acid (5 ng/ml), 10% Fetal calf serum, 2 mM L-glutamine, 25 mM Hepes and 50 ug/ml gentomycin. 50 ul of anti-CD40 mAb (0.2 ug/ml final) and 50 ul of hIL-4 (3 ng/ml final), 100 ul of TR2 mAb and 800 ul of cell suspension were added to were added to wells of a 48 well flat bottomed microtitre plates in triplicate. Controls included anti-CD40 and IL-4 alone, medium and IL-4 alone. Cells were cultured for 14 days at 37° C. in 5% CO2. 700 ul of supernantant from individual wells were harvested and stored at −20° C. Supernantants were assayed for human IgE using a human IgE ELISA detection assay. Briefly, Immunlon II ELISA plates were coated with Rabbit anti-human IgE antibody (Dako) in PBS containing 0.02% Sodium Azide at 4° C. overnight. Plates were washed 4 times with PBS containing 0.05% Tween 20 and 0.02% Sodium Azide (wash buffer). Plates were blocked for 60 minutes at 37° C. with PBS containing 0.1% gelatin and 0.02% Sodium Azide. After 4 washes using wash buffer, 100 ul of IgE standard or sample diluted in PBS containing 0.1% gelatin, 0.02% Sodium Azide and 0.5% Tween 20 (assay buffer) were added to wells in duplicate and incubated at 37° C. for 60 minutes. Plates were washed and incubated with monoclonal mouse anti-human IgE (Serotec) for 60 minutes at 37° C. in assay buffer. After washing the plates Goat anti-mouse antibody conjugated to alkaline phosphatase in assay buffer was added to each well and incubated at 37° C. for 60 minutes. Plates were washed and 100 ul of p-nitrophenyl phosphate (1 mg/ml) in substrate buffer containing diethanolamine was added to each well. Plates were allowed to develop and optical densities read at 405 nm on an ELISA plate reader.

In two separate experiments using 3 different donors, IgE production by PBMCs were inhibited by TR2 mAb 12C5 in a dose dependent manner. IC₅₀ values for the three donors were calculated to be <3 nM, 30 nM and 5 nM, respectively. These results indicate that TR2 mAb 12C5 is capable of inhibiting IgE production in response to hIL4 and anti-CD40 mAb. This suggests that the TR2 receptor is involved directly or indirectly in regulating PBMC IgE production in response to IL4 and anti-CD40 mAb.

What is claimed is:

1. A method of using antibodies specific for TR2 to treat pathological conditions comprising administering to a patient a therapeutic amount of TR2 antibody in need thereof.

2. A method of using antibodies specific to TR2 to treat a patient with pathological conditions comprising:
a) taking cells or tissues and incubating them with therapeutic amounts of antibodies ex-vivo; and
b) re-administering the cells or tissues back to the patient.

3. A method of using antibodies to TR2 for diagnosis of pathological conditions, for monitoring of patients with abnormalities in TR2 function, production or metabolism.

4. A method of claim 1, 2, and 3 in which pathological conditions are selected from the group consisting of systemic lupus erythomatosus (SLE); idiopathic thrombocytopenic purpura (ITP); rheumatoid arthritis (RA); multiple sclerosis (MS); psoriasis, inflammatory bowel disease (IBD); insulin dependent diabetes melititus (IDDM); allergic disorders, including asthma, allergic rhinitis, and atopic dermatitis; cancers, such as, lymphornas and leukemias; atherosclerosis; and viral infections, such as HSV infections and AIDS.

5. A method of using antibodies to TR2 for screening of compounds which agonizes or antagonizes TR2 comprising detecting the alteration of TR2 activity level in the presence of TR2 antibody and a candidate molecule which might otherwise be occupied by TR2 receptor ligand.

6. A method claim 1 in which pathological conditions are selected from allergic disorders including asthma, allergic rhinitis and atopic dermatitis.


SEQUENCE LISTING (4 sequences)

SEQ ID NO: 1 - DNA, 1704 bp, Homo sapien
gcacgagctgcctcccgcaggcgccacctgtgtcccccagcgccgctccacccagcaggcctgagcccctctctgctgccagacaccccctgctgcccactctcctgctgctcgggttctgaggcacagcttgtcacaccgaggcggattctctttctctttctctttctcttctggcccacagccgcagcaatggcgctgagttcctctgctggagttcatcctgctagctgggttcccgagctgccggtctgagcctgaggcatggagcctcctggagactgggggcctcctccctggagatccacccccaaaaccgacgtcttgaggctggtgctgtatctcaccttcctgggagccccctgctacgccccagctctgccgtcctgcaaggaggacgagtacccagtgggctccgagtgctgccccaagtgcagtccaggttatcgtgtgaaggaggcctgcggggagctgacgggcacagtgtgtgaaccctgccctccaggcacctacattgcccacctcaatggcctaagcaagtgtctgcagtgccaaatgtgtgacccagccatgggcctgcgcgcgagccggaactgctccaggacagagaacgccgtgtgtggttgcagcccaggccacttctgcatcgtccaggacggggaccactgcgccgcgtgccgcgcttacgccacctccagcccgggccagagggtgcagaagggaggcaccgagagtcaggacaccctgtgtcagaactgccccccggggaccttctctcccaatgggaccctggaggaatgtcagcaccagaccaagtgcagctggctggtgacgaaggccggagctgggaccagcagctcccactgggtatggtggtttctctcagggagcctcgtcatcgtcattgtttgctccacagttggcctaatcatatgtgtgaaaagaagaaagccaaggggtgatgtagtcaaggtgatcgtctccgtccagcggaaaagacaggaggcagaaggtgaggccacagtcattgaggccctgcaggcccctccggacgtcaccacggtggccgtggaggagacaataccctcattcacggggaggagcccaaaccactgacccacagactctgcaccccgacgccagagatacctggagcgacggctgaatgaaagaggctgtccacctggcggaaccaccggagcccggaggcttgggggctccaccctggactggcttccgtctcctccagtggagggagaggtggcgcccctgctggggtagagctggggacgccacgtgccattcccatgggccagtgagggcctggggcctctgttctgctgtggcctgagctccccagagtcctgaggaggagcgccagttgcccctcgctcacagaccacacacccagccctcctgggccaacccagagggccttcagaccccagctgtgtgcgcgtctgactcttgtggcctcagcaggacaggccccgggcactgcctcacagccaaggctggactgggttggctgcagtgtggtgtttagtggataccacatcggaagtgattttctaaattggatttgaattcggctcctgttttctatttgtcatgaaacagtgtatttggggagatgctgtgggaggatgtaaatatcttgtttctcctcaaaaaaaaaaaaaaaaaaaaaaaaaaaaaaaaaaa

SEQ ID NO: 2 - protein, 283 aa, Homo sapien
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 3 - DNA, 35 bp, Homo sapien
caggaattcgcagccatggagcctcctggagactg

SEQ ID NO: 4 - DNA, 53 bp, Homo sapien
ccatacccaggtaccccttccctcgatagatcttgccttcgtcaccagccagc